(19) United States
(12) Patent Application Publication
(10) Pub. No.: US 2012/0035127 A1
(43) Pub. Date: Feb. 9, 2012

(54) PROCESS FOR THE PRODUCTION OF A COMPOSITION, THE COMPOSITION AND THE USE THEREOF AS FOOD ADDITIVE

(75) Inventors: Dorothée Goffin, Malonne (BE); Michel Paquot, Noville-sur-Mehaigne (BE); Christophe Blecker, Forville (BE); Christelle Robert, Forrieres (BE)

(73) Assignee: Universite de Liege Gembloux Agro-Bio Tech, Liege (BE)

(21) Appl. No.: 13/144,473

(22) PCT Filed: Jan. 19, 2010
(86) PCT No.: PCT/EP2010/050596

§ 371 Date: Jul. 13, 2011

(30) Foreign Application Priority Data
Jan. 19, 2009 (EP) 09150869.7

(51) Int. Cl. A61K 31/715; A61P 29/00; A61P 1/00; A61P 35/00; A61P 3/04; A61P 37/02; A61P 9/00; A61P 3/10; A61K 31/7016; A61P 1/14

(52) U.S. Cl. 514/54; 514/53

(57) ABSTRACT

The present invention relates to a process for the production of a functional food additive, such as a prebiotic composition, comprising the steps of. (a) providing a plant based material wherein the plant is selected from the group consisting of cereals, legumes, tubers and mixtures thereof, wherein said plant based material comprises dietary fiber optionally starchy material and optionally glucose, or wherein said plant based material comprises starchy material, and optionally glucose; (b): (b1) hydrolyzing or tranglucosylating at least part of the dietary fiber into glucose and into at least one non-digestible oligosaccharide and optionally into at least one non-digestible polysaccharide, and optionally hydrolyzing and transglucosylating at least part of the starchy material to glucose and into at least one non-digestible oligosaccharide, or, (b2) hydrolyzing and transglucosylating at least part of the starchy material to glucose and into at least one non-digestible oligosaccharide, and optionally hydrolyzing at least part of the maltooligosaccharides produced in step (b2) into glucose; (c) oxidizing at least part of the total glucose, consisting of at least part of said gluconic acid and/or a salt thereof obtained in step (c); thereby obtaining a composition comprising dietary fiber and gluconic acid or a salt thereof, wherein said dietary fiber comprises at least one non-digestible oligosaccharide and optionally at least one non-digestible polysaccharide as defined in claim 1. This invention also relates to a functional food additive composition and the use thereof.

### FIELD OF THE INVENTION

The present invention relates to food processing. In particular the present invention relates to a process for the production of a functional food additive, such as a prebiotic composition. This invention also relates to the use of the functional food additive composition.

### BACKGROUND OF THE INVENTION

Food processing can be defined as methods and techniques used to transform raw ingredients produced by agriculture into food for human and animal consumption. In some processed foods, there is a lack of certain nutrients such as dietary fibers. This is often due to the refining process that has been used during the production of these foods.

The food ingredient industry offers a wide range of ingredients that can be added during food processing for different nutritional and/or techno-functional reasons. In order to be used in preparations for specific diets, some ingredients require low levels of specific components or contaminants, for example food for diabetic or food presenting low tooth decay risks often requires less than 1% by weight of glucose in the final product. Sometimes, some ingredients cannot be used because they modify the taste of the end product and make it unacceptable for the consumer e.g. sweet taste for some meat products or because they modify the characteristics, the properties and/or the behavior of the food product during its preparation e.g. presence of glucose coloring egg products during heating. The production of “organic” ingredients requires some purification techniques such as ion exchange resins or chromatographic resins that are not allowed by the “organic” food community. New techniques are to be found for the purification of these types of food ingredients.

Food manufacturers require ingredients that are of a well-defined composition, stable in time, and delivering at the smallest possible amount target characteristics.

Dietary fibers are an important component in the human and animal diet. Dietary fibers appear to have relevance in improving human and animal health. Extensive research is being conducted on nutritional and health benefits of new types of dietary fibers beyond the “classical” dietary fiber benefits. These potential benefits include the prebiotic effect or other benefits like alleviation of constipation, improved gut health, improved mineral absorption, improved lipid metabolism and a better regulation of glycemia/insulinemia levels.

Dietary fibers are naturally present in a wide variety of foods, particularly in vegetables and fruits. However, the world-wide consumption of dietary fibers remains well below the recommended daily values around 25-30 g/day. One of the reasons is the low consumption of fruits and vegetable, another one is that several types of “classical” dietary fibers greatly modify the taste and texture of foods and beverages, which is unacceptable for the consumer.

Thus, there is a continuing need for the preparation of food ingredients rich in dietary fibers. There is also a need to develop new types of dietary fibers such as non-digestible polysaccharides (NDP) and/or non-digestible oligosaccharides (NDO), that can be easily added as functional food additives to different types of foods and beverages without affecting the aspect, texture and taste of the product.

The sources of dietary fiber vary widely. Tubers, legumes, and cereals are generally recognized as a particularly interesting raw material for the production of dietary fibers

Cereal industries deliver by-products (bran, starch gluten separation by-products, corn wet milling starch by-products) that contain dietary fiber such as hemicellulose but that also contain high levels of starchy material. The extraction and purification of the dietary fibers often requires the separation and the elimination of the starchy material.

However, the production processes in the art, either provides product still too rich in starchy material, or results in the loss of soluble NDP and NDO during the process. For example some techniques of milling and air classification of brans allow the production of two fractions, one of which is poorer in starch. Nevertheless this fraction still contains high amounts of starchy material compared to the amounts of hemicellulose and other fibers. There is therefore also a need to provide food ingredients rich in NDP and/or NDO and poor in starchy material and/or glucose.

Several methods exist for the separation of the starchy material from the NDP and/or NDO. For example, one method involves the separation of starch by solubility in water: starch has a low solubility in cold water and can therefore be separated from NDP and/or NDO in solution by solubility difference. By centrifugation or by filtration, the insoluble material can be separated from the soluble material, but this step is difficult to scale up on an industrial scale. The solutions containing NDP and/or NDO and starch, are generally clogging for all type of filters, and the starch particles are too small to be efficiently separated by centrifugation.

Another known method involves Size Exclusion Chromatography (SEC) before starch hydrolysis: On SEC columns, the high molecular weight starch can be separated from smaller molecules as NDO and monosaccharides. However this method does not give satisfactory results because, on one hand the NDP is not separated from starch, and on the other hand glucose is not separated from the NDO.

Another known method involves the SEC after complete starch hydrolysis: on SEC columns glucose can be separated from larger molecules as NDO/NDP but usually, the separation is not accurate and the smallest molecule of NDO are not well separated from glucose. Furthermore, the SEC methods imply a very high dilution rates, which means high production costs to eliminate produced waste water.

Another known method involves alcoholic or acidic fermentation process: Starchy material and glucose can be eliminated from the solutions by fermentation using more or less specific micro-organisms consuming exclusively or preferably starchy material and/or glucose. The inconvenience of this method is the production of lots of different molecules, in small quantities, that negatively impact the quality of the product.

All the above described methods are associated with important losses of material and money, which make them poorly attractive. The different stages needed for the production of pure NDO and/or NDP from starch containing plants are complex and there is nowadays no way to produce them in an economically and environmentally acceptable manner. The problem remains unchanged for decennia's.

It is an object of the present invention to overcome or ameliorate at least one of the disadvantages of the prior art, or to provide a useful alternative.

### SUMMARY OF THE INVENTION

One object of the present invention is to provide a process for the production of a functional food additive that comprises several converting steps.

The process of the present invention is an elegant solution, from an economical, an environmental, a nutri-functional and a techno-functional point of view.

In a first aspect, the present invention provides a process as defined in the appended claims. In particular a process for the production of a composition, in particular a food composition, more in particular a food additive composition, yet more in particular a functional food additive composition, comprising the steps of:
a) providing a plant based material wherein the plant is selected from the group consisting of cereals, legumes, tubers and mixtures thereof, said plant based material comprising dietary fiber, and/or starchy material and optionally glucose,
b) converting at least part of the dietary fiber into glucose and/or into at least one non-digestible oligosaccharide (NDO) and/or at least one non-digestible polysaccharide (NDP); and/or converting at least part of the starchy material to glucose,
c) converting at least part of the total glucose, consisting of said optional glucose of step (a) and said glucose obtained in step (b), to gluconic acid and/or a salt thereof, and
d) removing at least part of said gluconic acid and/or a salt thereof obtained in step (c),
   thereby obtaining a composition comprising dietary fiber and optionally gluconic acid and/or a salt thereof,
   wherein said dietary fiber comprises at least one NDO selected from the group consisting of xylooligosaccharides, arabinoxylooligosaccharides, beta-glucan glucooligosaccharides, arabinogalactanoligosaccharides, isomaltooligosaccharides, xyloglucan oligosaccharides, galactomannan oligosaccharides, mannan oligosaccharides, cellulooligosaccharides, cellobiose and gentiooligosaccharides, and/or at least one NDP selected from the group consisting of beta-glucans, xylans, arabinoxylans, arabinogalactans, arabinogalactanpeptides, xyloglucans, mannans, galactomannans and cellulose.

In particular the present invention provides a process for the production of a composition comprising the steps of:
a) providing a plant based material wherein the plant is selected from the group consisting of cereals, legumes, tubers and mixtures thereof, wherein said plant based material comprises dietary fiber, optionally starchy material and optionally glucose, or wherein said plant based material comprises starchy material, and optionally glucose,
b) (b1) hydrolyzing or transglucosylating at least part of the dietary fiber into glucose and into at least one non-digestible oligosaccharide, optionally into at least one non-digestible polysaccharide, and optionally hydrolyzing and transglucosylating at least part of the optional starchy material to glucose and into at least one non-digestible oligosaccharide, or,
(b2) hydrolyzing and transglucosylating at least part of the starchy material to glucose and into at least one non-digestible oligosaccharide, and optionally hydrolyzing at least part of the maltooligosaccharides produced in step (b2) into glucose,
c) oxidizing at least part of the total glucose, consisting of said optional glucose of step (a) and said glucose obtained in step (b1) or (b2), to gluconic acid or a salt thereof, and
d) removing at least part of said gluconic acid or a salt thereof obtained in step (c);

thereby obtaining a composition comprising

gluconic acid or a salt thereof in a concentration by weight ranging between 11 and 50%;

dietary fiber wherein said dietary fiber comprises: at least one non-digestible oligosaccharide selected from the group consisting of xylooligosaccharides, arabinoxylooligosaccharides, beta-glucan glucooligosaccharides, arabinogalactanoligosaccharides, isomaltooligosaccharides, xyloglucan oligosaccharides, galactomannan oligosaccharides, mannan oligosaccharides, cellulooligosaccharides, cellobiose and gentiooligosaccharides in a concentration by weight ranging between 5 and 85%, and optionally at least one non-digestible polysaccharide selected from the group consisting of beta-glucans, xylans, arabinoxylans, arabinogalactans, arabinogalactanpeptides, xyloglucans, mannans, galactomannans and cellulose in a concentration by weight ranging between 0 and 20%,
optionally glucose in a concentration by weight ranging between from 0 to 2% ; and
optionally starchy material in a concentration by weight ranging between from 0 to 5%.

In an embodiment, said process further comprises the step of hydrolyzing at least part of non-digestible polysaccharide comprised in the dietary fiber into non-digestible oligosaccharide, wherein said hydrolyzing step is performed before, during, between, or after any of said steps (a) to (d).

In an embodiment, said steps (a), (b), (c) and (d) are performed consecutively.

In an embodiment, said step (b) and said oxidizing step (c) occur at least in part simultaneously.

In an embodiment, the process comprises removing less than 99% by weight of said gluconic acid or a salt thereof.

In an embodiment, the composition obtained comprises gluconic acid or a salt thereof in a concentration by weight ranging between 11 and 50%; arabinoxylooligosaccharides in a concentration by weight ranging between 5 and 85%, and optionally arabinoxylans in a concentration by weight ranging between 0 and 20%, optionally glucose in a concentration by weight ranging between from 0 to 2%; and optionally starchy material in a concentration by weight ranging between from 0 to 5%.

The process of this invention is particularly advantageous as it provides a new production process for a functional food additive that is economical and that respects the environment.

With this invention, it is no more necessary to discard digestible carbohydrates as such, which implies less burden on the environment. The invention also allows to keep a lot of raw materials and to improve the prebiotic quality of the final product by synergy with gluconic acid resulting from glucose conversion.

The present invention also encompasses the composition directly obtained by the process according to the invention.

In a second aspect, the present invention provides a composition suitable for a functional food additive composition as defined in the appended claims, and preferably as a prebiotic composition, also herein referred to as “the composition”.

The composition according to the present invention comprises:
gluconic acid and/or a salt thereof in a concentration by weight ranging between 1 and 60%;
at least one NDO selected from the group consisting of xylooligosaccharides, arabinoxylooligosaccharides, beta-glucan glucooligosaccharides, arabinogalactanoligosaccharides, isomaltooligosaccharides, xyloglucan oligosaccharides, galactomannan oligosaccharides, mannan oligosaccharides, cellulooligosaccharides, cellobiose and gentiooligosaccharides in a concentration by weight ranging between 1 and 95%, and/or
at least one NDP selected from the group consisting of beta-glucans, xylans, arabinoxylans, arabinogalactans, arabinogalactanpeptides, xyloglucans, mannans, galactomannans and cellulose in a concentration by weight ranging between 1 and 95%.

One embodiment of the present invention concerns a composition wherein the concentration by weight of gluconic acid and/or a salt thereof is ranging between 1% and 60%, preferably between 11 and 50%, and most preferably between 20% and 40% by weight; the concentration by weight of NDO is ranging between 1 and 95%, preferably between 5% and 95%, preferably between 5% and 85%, and most preferably between 10 and 80%, and the concentration by weight of NDP is ranging between 0 and 95%, for example between 0 and 20%, preferably between 1 and 95%, preferably between 5 and 80% and most preferably between 10 and 50%.

In a preferred embodiment, the composition according to the invention comprises:
gluconic acid or a salt thereof in a concentration by weight ranging between 11 and 50%;
at least one non-digestible oligosaccharide selected from the group consisting of xylooligosaccharides, arabinoxylooligosaccharides, beta-glucan glucooligosaccharides, arabinogalactanoligosaccharides, isomaltooligosaccharides, xyloglucan oligosaccharides, galactomannan oligosaccharides, mannan oligosaccharides, cellulooligosaccharides, cellobiose and gentiooligosaccharides in a concentration by weight ranging between 5 and 85%, and optionally
at least one non-digestible polysaccharide selected from the group consisting of beta-glucans, xylans, arabinoxylans, arabinogalactans, arabinogalactanpeptides, xyloglucans, mannans, galactomannans and cellulose in a concentration by weight ranging between 0 and 20%;
optionally glucose in a concentration by weight ranging between 0 and 2% and
optionally starchy material in a concentration by weight ranging between 0 and 5%.

In an embodiment, said non-digestible oligosaccharide and non-digestible polysaccharide in said composition are originated from plant based material wherein the plant is selected from the group consisting of cereals, legumes, tubers and mixtures thereof.

In an embodiment, said non-digestible oligosaccharide in said composition is selected from the group consisting of arabinoxylooligosaccharides, xylooligosaccharides, beta-glucan glucooligosaccharides, cellobiose, organic isomaltooligosaccharides and mixtures thereof.

In an embodiment, said non-digestible polysaccharide in said composition is selected from the group consisting of arabinoxylans, arabinogalactans, arabinogalactanpeptides, beta-glucans, and mixtures thereof.

In an embodiment, the concentration by weight of gluconic acid or a salt thereof in said composition is ranging between 11 and 50%, the concentration by weight of non-digestible oligosaccharide is ranging between 10 and 50%, and the concentration by weight of non-digestible polysaccharide is ranging between 0 and 20%.

In a preferred embodiment, the composition comprises gluconic acid or a salt thereof; arabinoxylooligosaccharides and optionally arabinoxylans, preferably said composition comprises gluconic acid or a salt thereof in a concentration by weight ranging between 15 and 50%; arabinoxylooligosaccharides in a concentration by weight ranging between 5 and 50% and optionally arabinoxylans in a concentration by weight ranging between 0 and 20%.

In an embodiment, the composition according to the invention further comprises inulin and/or oligofructose.

In an embodiment, the gluconic acid salt in said composition is selected from sodium gluconate, potassium gluconate, calcium gluconate, magnesium gluconate, iron gluconate, selenium gluconate, copper gluconate or zinc gluconate.

The compositions according to the present invention may be formulated as a powder, a liquid or a dispersion of a powder in a liquid.

The compositions according to the invention are particularly useful as food additive, in particular as functional food additive and preferably as prebiotic composition.

The present invention therefore also concerns the use of a composition according to the invention, for providing a technical, nutritional and/or health benefit to a human being or animal in need thereof.

In an embodiment, the present composition can be used for the selective stimulation of the growth and/or activity of the gastro-intestinal microflora. In a further embodiment, said composition can also be used for the alleviation of constipation, for improving gut health, for improving mineral absorption, for improving lipid metabolism, and/or for a better regulation of glycemia/insulinemia. The present composition can also be used for the reduction of the risk of heart disease, diabetes and/or metabolic syndrome, cancer prevention, positive impact on hepatic encephalopathy, glycemia/insulinemia regulation, immunomodulation, inflammation reduction. The present composition is also particularly useful for improving satiety.

The present invention also encompasses the use of a composition according to the invention, containing gluconate salts for providing cations to a human being or an animal in need thereof.

In a further aspect, the present invention provides a method for preparing a food product using a composition according to the invention. In particular the present invention provides a method for preparing a food product, such as a beverage, comprising the steps of:

a. providing a composition according to the invention, and

b. formulating said composition into a food product.

In a further embodiment, this invention provides a food product or a beverage that contains the composition according to present invention.

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

### DESCRIPTION OF THE INVENTION

When describing the process and compositions of the present invention, the terms used are to be construed in accordance with the following definitions, unless a context dictates otherwise.

As used herein the term “comprising” should not be interpreted as being restricted to the means listed thereafter; i.e. it does not exclude other elements or steps.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

As used in the specification and the appended claims, the singular forms “a”, “an,” and “the” include plural referents unless the context clearly dictates otherwise. By way of example, “a NDO” means one NDO or more than one NDO.

As used herein, the term “monosaccharide” refers to a single sugar unit which is the building block of oligo- and polysaccharides. Non-limiting examples of monosaccharide include glucose, fructose, xylose, arabinose, galactose, mannose and the like.

As used herein, the term “carbohydrate” refers to a polyhydroxy-aldehyde (aldose) or ketone (ketose) or to a substance which yields one of these substances by hydrolysis.

As used herein, the terms “dietary fiber” or “fiber” refer to the edible parts of plants or analogous carbohydrates that are resistant to digestion and absorption in the human small intestine with complete or partial fermentation in the large intestine. Dietary fiber includes non-digestible polysaccharides, non-digestible oligosaccharides, lignin, and associated plant substances. (Cereal Foods World, 2001, 46, 112-126). In the context of the present invention dietary fiber or fiber refer to non-digestible polysaccharides and/or non-digestible oligosaccharides.

As used herein, the terms “degree of polymerization” or “(DP)” refers to the number of monosaccharide residues present in an oligo- or polysaccharide.

As used herein, the term “polysaccharide” refers to a carbohydrate composed of a large number (DP>10) of monosaccharides that are linked by glycosidic linkages. Non-limiting examples of naturally occurring polysaccharides are plant cell wall polysaccharides such as cellulose, pectins, arabinans/arabans, arabinoxylans, xylans, arabinogalactans, xyloglucans, betaglucans or other polysaccharides like starches, galactomannans, mannans, arabinogalactans, and fructans.

As used herein, the term “oligosaccharide” refers to a carbohydrate composed of a limited number of monosaccharides that are linked by glycosidic linkages; the DP generally ranging from 2 to 10. Non-limiting examples of naturally occurring oligosaccharides are saccharose, cellobiose, raffinose, fructo-oligosaccharides, and galacto-oligosaccharides.

As used herein, the term “starchy material” refers to starch and/or its hydrolysis products, such as dextrins, maltodextrins, maltose and/or blends of all or some of them. Usually, starchy material can be hydrolyzed to monosaccharides in the upper part of the gastrointestinal tract by first an acid action in the stomach and then by endogenous enzymes from the intestinal tract. The resulting monosaccharides are then absorbed in the blood. As used herein, the term “starch” refers to a polysaccharide carbohydrate consisting of a large number of glucose monosaccharide units joined together by glycosidic bonds. Most plant seeds and tubers contain starch which is predominantly present as amylose and amylopectin.

As used herein, the term “non-digestible oligosaccharide (NDO) and non-digestible polysaccharide (NDP)” refer to complex carbohydrates that escape digestion and/or absorption in the upper digestive tract of humans mainly due to the configuration of their osidic bonds. They thus arrive in the large intestine where some of them can be partially or totally fermented by the endogenous microflora. This fermentation process generates gases and/or short-chain fatty acids like for instance acetate, propionate and butyrate.

As used herein, the term “plant based material” refers to vegetable material originated from plants, comprising but not limited to cereals, legumes, tubers. Most of these plants contain starch.

As used herein, the term “cereals” refer to cereal plants including but not limited to wheat, oat, rye, barley, sorghum, maize, rice, millet, sorghum, and triticale.

As used herein, the term “legumes” refer to plants of the Leguminosae family including but not limited to pea, bean, lentil, soya and lupin.

As used herein, the term “tubers” refer to stem tuber or root tuber plants including but not limited to potato.

As used herein, the term “prebiotic ” refers to a non-digestible (or poorly digestible) food ingredient that beneficially affects the host by selectively stimulating the growth and/or activity of one or a limited number of bacteria in the colon, and thus improves host health (Gibson & Roberfroid, 1995, J Nutr 125, 1401-1412.).

As used herein, the term “prebiotic effect” refers to the selective stimulation of the growth and/or activity of one or a limited number of bacteria in the colon, thus improving host health. In the context of the two latter definitions, “host” has to be understood as a human being or an animal.

As used herein, the term “food” encompasses food for human or animal consumption.

As used herein, the term “food additive” refers to an ingredient, additive, component or supplement suitable for incorporation in human or animal food.

As used herein, the term “functional food additive” refers to an ingredient, additive, component or supplement suitable for incorporation in human or animal food conferring a technical, nutritional and/or health benefit to the host like for example a prebiotic effect and/or another nutritional/health benefit closely related to the selective stimulation of some colonic bacteria such as for the alleviation of constipation, for an improved gut health, for an improved mineral absorption, an improved lipid metabolism, and a better regulation of glycemia/insulinemia and thus reduction of the risk of heart disease, diabetes and/or metabolic syndrome, cancer prevention, positive impact on hepatic encephalopathy, immunomodulation, inflammation reduction, or improved satiety.

As used herein, the terms “isomaltooligosaccharides” or “(IMO)” refer to oligosaccharides of glucose, possessing specific α linkages. To be considered as an IMO, the gluco-oligosaccharide should have at least one of these specific types of linkage between 2 glucose monomers: α (1-6) (classical IMO), α (1-2) (koji-family) or α (1-3) (nigero-family). These linkages are conferring to IMO their low or non digestibility by human enzymes. The most frequent bond in the IMO is the α (1-6) bond between glucoses. The most frequent IMO are: isomaltose (α-D-Glcp-(1→6)-α-D-Glcp), panose (α-D-Glcp-(1→6)-α-D-Glcp-(1→4)-D-Glcp) and isomaltotriose (α-D-Glcp-(1▪6)-α-D-Glcp-(1▪6)-D-Glcp). In an embodiment of the present invention, IMO is preferably organic IMO, i.e. IMO as organic food or organic ingredient.

As used herein, the terms “arabinoxylooligosaccharides” or “(AXOS)” refer to oligosaccharides of xylose units linked by β (1-4) bonds and substituted to varying extents on O-2 and/or O-3 by arabinose units. Ferulic acid, galactose and/or glucuronic acid may also be present in the oligosaccharide structure.

As used herein, the term “gluconic acid” refers to an oxidative product of glucose, wherein the C1 hydroxyl group of glucose is oxidized to a carboxylic acid group. Gluconic acid is a monomeric non-carbohydrate organic acid. Gluconate may be defined as any possible salt of gluconic acid, whatever its countercation such as but not limited to sodium, potassium, calcium, magnesium, iron, selenium, copper or zinc. The composition according to the invention comprising the gluconate salts has the advantage of providing cations at the intestine level in a more bio-available form. The present invention therefore encompasses the use of a composition as defined herein for providing cations to a human being or an animal in need thereof.

As used herein, when not specified otherwise, the term “gluconic acid” comprises gluconic acid, and/or a salt thereof (gluconate) and/or any hydrated, dehydrated or solvate form thereof.

As used herein, the terms “ organic food ”or “organic ingredient” refers to a food or an ingredient that is produced following the prescriptions of the “organic or bio community and life style” that is well known for their refusal of non-natural fertilizers, pesticides, purification techniques, packaging techniques, etc. . . .

As used herein, the term “reaction medium ” refers to the mixture originated from plant based material wherein the plant is selected from the group consisting of cereals, legumes, tubers and mixtures thereof, said plant based material comprising dietary fiber and/or starchy material and optional glucose, and/or their derivatives products. According to the present invention the starchy material and optional glucose need to be at least partially removed from the reaction medium. Non-limiting examples of reaction medium could be for example, the raw material used for the process according to the invention. It can be for example the overflow of a three-phase decanter running in a wheat starch-producing unit, or it could be process water from a starch-gluten separation.

As used herein, the expression “%” refers to “% by weight expressed on dry matter”. The % can be calculated on the total reaction medium or composition according to the present invention.

According to embodiment of the present invention, a functional food additive composition is prepared using a process comprising the steps of:
a) providing a plant based material wherein the plant is selected from the group consisting of cereals, legumes, tubers and mixtures thereof, wherein said plant based material comprises fiber, optionally starchy material and optionally glucose, preferably said plant based material comprises starchy material, fiber, and optionally glucose; or wherein or wherein said plant based material comprises starchy material, and optionally glucose,
b) (b1) hydrolyzing or transglucosylating part or all of the fiber into glucose and into at least one NDO and optionally into at least one NDP, and optionally hydrolyzing and transglucosylating part or all of the starchy material to glucose and into at least one NDO, or,
(b2) hydrolyzing and transglucosylating at least part of the starchy material to glucose and into at least one NDO, and optionally hydrolyzing at least part of the maltooligosaccharides produced in step (b2) into glucose,
c) oxidizing part or all of the total glucose, consisting of said optional glucose of step (a) and said glucose obtained in step (b1) or (b2), to gluconic acid or a salt thereof, and
d) removing part of said gluconic acid or a salt thereof obtained in step (c);
   thereby obtaining a composition comprising 11 to 50% by weight of gluconic acid or a salt thereof, fiber wherein said fiber comprises: 5 to 85% by weight of at least one NDO selected from the group consisting of xylooligosaccharides, arabinoxylooligosaccharides, beta-glucan glucooligosaccharides, arabinogalactanoligosaccharides, isomaltooligosaccharides, xyloglucan oligosaccharides, galactomannan oligosaccharides, mannan oligosaccharides, cellulooligosaccharides, cellobiose and gentiooligosaccharides, and optionally 0 to 20% by weight of at least one NDP selected from the group consisting of beta-glucans, xylans, arabinoxylans, arabinogalactans, arabinogalactanpeptides, xyloglucans, mannans, galactomannans and cellulose,
   and less than 2% glucose and less than 5% starchy material.

One embodiment of the present invention concerns a process wherein said dietary fiber comprises at least NDO and at least one NDP.

The process according to the present invention is an efficient and attractive production process for the production of a functional food additive composition, having specific unexpected properties being economical and respecting the environment.

A person skilled in the art will understand that the steps of the present process can be done consecutively and that, in some cases some steps can be done, totally or partially, simultaneously, as it can be the case for the converting (hydrolyzing or tranglucosylating) step (b), and the converting (oxidizing) step (c). One embodiment of the present invention concerns a process wherein process steps (a) to (d) are performed consecutively. In another embodiment, the converting (oxidizing) step (c) and the removing step (d) may occur at least in part simultaneously.

In an embodiment, the present invention provides a process for the production of a composition comprising the steps of:

(a) providing a plant based material wherein the plant is selected from the group consisting of cereals, legumes, tubers and mixtures thereof, said plant based material comprising starchy material, dietary fiber and optionally glucose; or wherein said plant based material comprises starchy material, and optionally glucose,

(b) converting (hydrolyzing and transglucosylating) at least part of said starchy material to glucose and into at least one NDO, and optionally hydrolyzing at least part of the maltooligosaccharides produced in step (b) into glucose,

(c) converting (oxidizing) at least part of the total glucose, consisting of said optional glucose of step (a) and said glucose obtained in step (b), to gluconic acid and/or a salt thereof, and

(d) removing at least part of said gluconic acid and/or a salt thereof obtained in step (c);
thereby obtaining a composition comprising 11 to 50% by weight gluconic acid or a salt thereof, and dietary fiber, and wherein said dietary fiber comprises 5 to 85% by weight of at least one NDO selected from the group consisting of xylooligosaccharides, arabinoxylooligosaccharides, beta-glucan glucooligosaccharides, arabinogalactanoligosaccharides, isomaltooligosaccharides, xyloglucan oligosaccharides, galactomannan oligosaccharides, mannan oligosaccharides, cellulooligosaccharides, cellobiose, and gentiooligosaccharides, and optionally 0 to 20% by weight of at least one NDP selected from the group consisting of beta-glucans, xylans, arabinoxylans, arabinogalactans, arabinogalactanpeptides, xyloglucans, mannans, galactomannans and cellulose, and from 0 to 2% by weight of glucose and from 0 to 5% by weight of starchy material.

In an embodiment, said starchy material is at least partially converted to non-digestible oligosaccharide before, during, between, or after any of said steps (a) to (d). Preferably said starchy material is at least partially converted to at least one NDO during step (b). In a preferred embodiment, said starchy material is at least partially converted to IMO during step (b).

According to embodiment of the present invention, a functional food additive composition is prepared using a process comprising the steps of:
a) providing a plant based material wherein the plant is selected from the group consisting of cereals, legumes, tubers and mixtures thereof, wherein said plant based material comprises fiber, optionally starchy material and optionally glucose,
b) hydrolyzing or transglucosylating part or all of the fiber into glucose and into at least one NDO and optionally into at least one NDP, and optionally hydrolyzing and transglucosylating part or all of the starchy material into glucose and into at least one non-digestible oligosaccharide,
c) oxidizing part or all of the total glucose, consisting of said optional glucose of step (a) and said glucose obtained in step (b), to gluconic acid or a salt thereof, and
d) removing part of said gluconic acid or a salt thereof obtained in step (c);
   thereby obtaining a composition comprising 11 to 50% by weight of gluconic acid or a salt thereof, fiber wherein said fiber comprises: 5 to 85% by weight of at least one NDO selected from the group consisting of xylooligosaccharides, arabinoxylooligosaccharides, beta-glucan glucooligosaccharides, arabinogalactanoligosaccharides, isomaltooligosaccharides, xyloglucan oligosaccharides, galactomannan oligosaccharides, mannan oligosaccharides, cellulooligosaccharides, cellobiose and gentiooligosaccharides, and optionally 0 to 20% by weight of at least one NDP selected from the group consisting of beta-glucans, xylans, arabinoxylans, arabinogalactans, arabinogalactanpeptides, xyloglucans, mannans, galactomannans and cellulose,
   and from 0 to 2% by weight of glucose and from 0 to 5% by weight of starchy material.

In another embodiment, the present invention provides a process for the production of a composition comprising the steps of:

(a) providing a plant based material wherein the plant is selected from the group consisting of cereals, legumes, tubers and mixtures thereof, said plant based material comprising starchy material and optionally glucose;

(b) converting (hydrolyzing and transglucosylating) at least part of said starchy material to at least one NDO and glucose, and optionally hydrolyzing at least part of the maltooligosaccharides produced in step (b) into glucose,

(c) converting (oxidizing) at least part of the total glucose, consisting of said optional glucose of step (a) and said glucose obtained in step (b), to gluconic acid or a salt thereof, and

(d) removing at least part of said gluconic acid or a salt thereof obtained in step (c);
thereby obtaining a composition comprising gluconic acid or a salt thereof, at least one NDO, optionally at least one NDP, wherein said at least one NDO is selected from the group consisting of xylooligosaccharides, arabinoxylooligosaccharides, beta-glucan glucooligosaccharides, arabinogalactanoligosaccharides, isomaltooligosaccharides, xyloglucan oligosaccharides, galactomannan oligosaccharides, mannan oligosaccharides, cellulooligosaccharides, cellobiose, and gentiooligosaccharides, and said at least one NDP is selected from the group consisting of beta-glucans, xylans, arabinoxylans, arabinogalactans, arabinogalactanpeptides, xyloglucans, mannans, galactomannans and cellulose. Preferably, said composition comprises from 11 to 50% by weight of gluconic acid or a salt thereof, from 5 and 85% by weight of NDO and optionally from 0 and 20% by weight of NDP, from 0 and 2% by weight of glucose and from 0 and 5% of starch.

In another embodiment, the present invention provides a process for the production of a composition comprising the steps of:

(a) providing a plant based material wherein the plant is selected from the group consisting of cereals, legumes, tubers and mixtures thereof, said plant based material comprising dietary fiber and optionally glucose;

(b) converting (hydrolyzing or tranglucosylating) at least part of said dietary fiber into glucose and/or into at least one non-digestible oligosaccharide and/or at least one non-digestible polysaccharide, preferably hydrolyzing or tranglucosylating at least part of said dietary fiber into glucose and into at least one NDO and optionally into least one NDP,

(c) converting (oxidizing) at least part of the total glucose, consisting of said optional glucose of step (a) and said glucose obtained in step (b), to gluconic acid or a salt thereof, and

(d) removing at least part of said gluconic acid and/or a salt thereof obtained in step (c);
thereby obtaining a composition comprising dietary fiber, and gluconic acid or a salt thereof, wherein said dietary fiber comprises:
at least one NDO selected from the group consisting of xylooligosaccharides, arabinoxylooligosaccharides, beta-glucan glucooligosaccharides, arabinogalactanoligosaccharides, isomaltooligosaccharides, xyloglucan oligosaccharides, galactomannan oligosaccharides, mannan oligosaccharides, cellulooligosaccharides, cellobiose, and gentiooligosaccharides, and optionally at least one NDP selected from the group consisting of beta-glucans, xylans, arabinoxylans, arabinogalactans, arabinogalactanpeptides, xyloglucans, mannans, galactomannans and cellulose. Preferably, said composition comprises from 11 to 50% by weight of gluconic acid or a salt thereof, from 5 and 85% by weight of NDO and optionally from 0 and 20% by weight of NDP, from 0 and 2% by weight of glucose and from 0 and 5% of starch.

The process according to the invention may also comprise a step wherein at least one NDP comprised in the plant based material is at least partially converted (hydrolyzed) to NDO before, during, between, or after any one of the steps (a) to (d).

Step (a) of the present process comprises providing a plant based material comprising dietary fiber, and optionally starchy material and optionally glucose, or a plant based material comprising starch material and optionally glucose. In an embodiment, step (a) of the present process comprises providing a plant based material comprising starchy material, dietary fiber, and optionally glucose. In another embodiment, step (a) of the present process comprises providing a plant based material comprising starchy material, and optionally glucose. In another embodiment, step (a) of the present process comprises providing a plant based material comprising dietary fiber, and optionally glucose.

Different raw materials, that contain starchy material and/or dietary fiber and optional glucose, can be provided in this process step. For the purpose of this invention, the raw material will be originated from plants (plant based material), wherein the plant will be selected from the group consisting of cereals, legumes, tubers and all possible mixtures thereof. Non-limiting examples of suitable plant based material includes derivatives of cereals, legumes and tubers, such as cereal bran, starch-gluten separation by-products, corn wet milling starch by-products, starch industry by-products, starch, cellulose, hemicellulose, lignocellulosic material, etc. and mixtures thereof.

In an embodiment, in a preliminary phase of step (a), starch or starchy material comprised in the plant based material may be converted (hydrolyzed and transglucosylated) at least partially to dietary fiber, preferably to NDO. In an embodiment, this preliminary converting step comprises treating the starchy material with an enzyme such as an alpha-amylase, and then treating the reaction product of said amylase hydrolysis with a beta-amylase and a transglucosidase.

In another embodiment, in a preliminary phase of step (a), the NDP comprised in the plant based material may be converted (hydrolyzed or tranglucosylated) at least partially to NDO and/or NDP.

Suitable non-limiting examples of such plant based materials originating from different industries/technologies that can be used in step (a) are: the different process waters of the starch-gluten separation industries, or the overflow of a three-phase decanter running in a wheat starch production unit, or the suspension obtained after water immersion of cereal brans, for the production of arabinoxylans, arabinoxylooligosaccharides, and/or beta-glucan containing functional food additives. Another suitable example is the mixture of maltodextrins, glucose and IMO, wherein said mixture is obtained during the production of IMO from starch, for the production of IMO and preferably organic IMO.

In an embodiment, a pre-treatment of the raw materials can be performed, if required, before being processed according to the present invention. This pre-treatment step may comprise the physical separation of the most important part of the solid material. This pre-treatment can be useful to already separate (remove) part of the starchy material. Suitable different techniques can be used for this pretreatment such as centrifugation, microfiltration, centrifugal decantation, filtration, sedimentation, etc. In some cases, the hydrolytic action of some proteases (particularly alkaline proteases) before the physical separation step can be useful for a better cleaning of the raw material.

Step (b) of the present process comprises converting (hydrolyzing or tranglucosylating) at least part of the dietary fiber into glucose and/or (preferably and) into at least one non-digestible oligosaccharide and/or at least one non-digestible polysaccharide, and/or converting at least part of the starchy material to glucose and optionally into NDO, and optionally hydrolyzing at least part of the maltooligosaccharides (produced during the hydrolysis of the starchy material) to glucose. In an embodiment, said step (b) of the present process comprises converting (hydrolyzing) at least part of said starchy material to glucose. In an embodiment, said step (b) of the present process comprises (hydrolyzing and transglucosylating) at least part of said starchy material to glucose and NDO. In an embodiment, step (b) comprises hydrolyzing and transglucosylating at least part of the starchy material to glucose and into at least one NDO, and optionally hydrolyzing at least part of the maltooligosaccharides produced in said step to glucose.

This step of converting the starchy material and/or the dietary fiber can be performed enzymatically or chemically. Preferably, said step (b) is performed enzymatically.

In an embodiment, the pH and temperature of the plant based material of step (a) (also referred herein as “raw material”), optionally after pre-treatment, is adjusted such as to have an efficient conversion of the starchy material to glucose. The usual techniques of the dextrose production industries give suitable guidelines for this process step. For example, starch can be gelatinized and a jet cooker is for that purpose a suitable equipment therefor.

When the converting step is performed enzymatically, non-limiting suitable enzymes for the conversion of the starchy material can be selected from amylases and gluco-amylases, such as but not limited to alpha-amylase, beta-amylase, amyloglucosidase and alpha-glucosidase. Cellulase is a non-limiting example of a suitable enzyme for the conversion of dietary fiber such as cellulose.

In an embodiment, said converting step (b) comprises treating the starchy material with an alpha-amylase. In another embodiment, said converting step (b) comprises treating the starchy material with a beta-amylase. In another embodiment, said converting step (b) comprises treating said starchy material with a glucoamylase.

In an embodiment, said converting step (b) comprises first treating the starchy material with an alpha-amylase, and then treating the obtained reaction product with a beta-amylase, optionally in the presence of a transglucosidase. The reaction product of the beta-amylase treatment can be further treated with a glucoamylase or a transglucosidase.

The converting step (b) using amylase can be performed at a temperature ranging from 35° C. to 100° C., preferably at a temperature ranging from 40 to 95° C.

In an embodiment, said converting step (b) further comprises treating the obtained reaction product with other enzymes such as tranglucosidase, glucoamylase, alcalase, and/or alkaline protease. In an embodiment, said converting step (b) further comprises treating the obtained product with glucoamylase, alcalase, and alkaline protease.

The use of a chemical process for the converting step (b) is also possible in the present invention. For example, acidification can be performed using an acid such as hydrochloric acid and operating at convenient temperature and optimum pH to allow the hydrolysis of the most part of the starchy material to glucose. For example, when this step is chemically performed at pH of 1.6 and at 125° C., under about 17 bar pressure, a starchy solution can reach a dextrose equivalent of 85 DE after 10 minutes.

The degree of advancement of converting step (b) can determine part of the glycemic index (GI) of the end product. The more starchy material escapes this conversion step to glucose, the more starchy material will be left in the end product, and the higher will be the GI of the end product.

In an embodiment, during this converting step (b) at least 50% by weight of the starchy material is converted to glucose. In a preferred embodiment, at least 70% by weight of the starchy material is converted to glucose. Most preferably at least 90% by weight of the starchy material is converted to glucose.

In another embodiment, during this converting step (b) at least 50% by weight of the dietary fiber is converted to NDO and/or glucose. In a preferred embodiment, at least 70% by weight of the plant based material is converted to NDO and/or glucose. Most preferably at least 90% by weight of the plant based material is converted to NDO and/or glucose.

Step (c) of the present process comprises converting (oxidizing) at least part of glucose to gluconic acid and/or a salt thereof.

The converting step can be performed chemically, electrochemically, isoelectrochemically, enzymatically or microbiologically. When performed microbiologically said reaction can be performed using for example Aspergillus niger and/or Gluconobacter oxydans and the like).

One embodiment of the present invention concerns a process wherein step (c) of converting glucose to gluconic acid and/or a salt thereof is performed enzymatically. Suitable enzymatic conversion of glucose to gluconic acid is described below and illustrated in scheme 1.

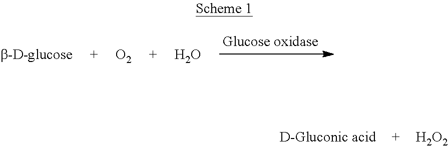

A suitable enzyme for this enzymatic conversion is glucose oxidase (GOX). Glucose oxidase is commercially available and its operating conditions are well known (e.g.: Gluzyme™ from Novo Nordisk). A schematic representation of the enzymatic conversion of glucose to gluconic acid by glucose oxidase is shown in Scheme 1. The reaction is a two step-reaction, wherein the first step occurs in presence of GOX and comprises the conversion of β-D-Glucose (C₆H₁₂O₆) to D-gluconic acid (C₆H₁₂O₇) in aqueous conditions.

The second step comprises the reduction of O₂ to hydrogen peroxide. Hydrogen peroxide is one of the products of the glucose oxidation. The peroxide is eliminated for example by using catalase in the reaction medium. Usually some catalase is present in the commercially available glucose oxidase preparations. A high catalase dosage in the reaction medium will also have a positive effect on the glucose oxidation reaction. Suitable catalase for use in the glucose oxidation step can be selected from the different available commercial catalases. Alternative suitable techniques to degrade hydrogen peroxide include the use of reducing agents (e.g sodium bisulfite), metal catalysts, or UV lights. Sodium bisulfite can advantageously be added very early in the process to act as antioxidant, and to protect the solution from excessive color formation, that occurs during heating and oxygen pick-up.

Oxidation of glucose to gluconic acid is preferably performed in the presence of excess of oxygen. Oxygen can be dissolved in the reaction medium. Preferably, air or oxygen is dispersed in the reaction medium during the entire reaction time.

The pH is preferably adjusted and maintained in order to maintain the activity of the glucose oxidase and the catalase at the optimum level. This can be performed through the use of a suitable buffer solution or by adding an alkaline agent such as sodium hydroxide, calcium carbonate or calcium hydroxide. The use of calcium carbonate or calcium hydroxide, has the advantages of regulating the pH, but will also result in a precipitation of part of the produced gluconic acid as gluconate, that can be further separated by filtration, as example. Other cations can be used to precipitate gluconic acid such as magnesium, selenium, zinc, copper or iron. Alternatively, the gluconate obtained after precipitation is converted to another salt by salt exchange process.

In one embodiment of this invention at least 50% by weight of the total glucose is converted to gluconic acid, resulting in a low glycemic index of the produced functional food additive.

In a preferred embodiment of this invention at least 70% by weight of the total glucose is converted to gluconic acid, resulting in a low glycemic index of the produced functional food additive.

In an embodiment, during this converting step (c), at least 50%, preferably at least 70% and most preferably at least 90% by weight of the total glucose is converted to gluconic acid and/or a salt thereof.

This allows the production of composition having tailored glycemic index, and when 90% of glucose is converted, composition with very low glycemic index are produced.

Step (d) of the present process comprises removing at least part of the produced gluconic acid and/or a salt thereof.

A part of the gluconic acid and/or a salt thereof can be separated from the reaction medium by using one of the following techniques, such as but not limited to ion exchange, electro-dialysis, or precipitation.

In step (c), the elimination of gluconic acid based on the precipitation of calcium gluconate has already been mentioned.

One embodiment of the present invention concerns a process wherein step (d) comprising removing less than 99% by weight of gluconic acid produced in step (c) and preferably less than 80% and most preferably less than 60% by weight of gluconic acid and/or a salt thereof.

In a preferred embodiment, calcium gluconate is precipitated at a temperature of about 15° C., and separated (e.g. by filtration, centrifugation, decantation, . . . ) from the reaction medium. In this embodiment, the gluconic acid is removed such as to result in a residual amount of gluconic acid that does not exceed 30% by weight in the composition.

In another embodiment, the removal of part or all of the gluconic acid, is performed by a demineralisation technique using ion exchange resins or electro-dialysis. Gluconic acid can be selectively eliminated by using a simple strong anionic exchange resin. Cations and gluconic acid can be simultaneously removed by using a combined strong-cation, weak-anion exchange resin. For the last purpose, electro-dialysis is also convenient.

The person skilled in the art will understand that, after completing detailed process steps (a) to (d), the reaction medium can be submitted to additional treatments in order to become a marketable stable functional food additive. Without being exhaustive, one or more further treatments can be applied in order to deliver a functional food additive with improved taste performance, providing less after taste, and containing less impurities, such as filtration, ultrafiltration, active carbon treatment, water evaporation, pasteurisation, sterilisation and spray drying.

The production of functional food additives according to the present invention optionally comprises the conversion of at least part of NDP comprised in the plant based material to NDO. This conversion step can take place at different moments before, during, between or after any one of steps (a-d).

It is the choice of the person skilled in the art, to make the most appropriate combination of the steps, so that his objectives are obtained in an optimal way.

An advantage of the present invention is that it allows the production of purified IMO as organic ingredients, because the transformation of glucose to gluconic acid followed by precipitation of gluconate cancels the need for other purification techniques such as ion exchange that are not allowed for organic food production.

The present invention also encompasses the composition directly obtained by the process according to the invention. The present invention therefore encompasses a composition comprising 11 to 50% by weight gluconic acid and/or a salt thereof, and dietary fiber, wherein said dietary fiber comprises:

at least one NDO selected from the group consisting of xylooligosaccharides, arabinoxylooligosaccharides, beta-glucan glucooligosaccharides, arabinogalactanoligosaccharides, isomaltooligosaccharides, xyloglucan oligosaccharides, galactomannan oligosaccharides, mannan oligosaccharides, cellulooligosaccharides, cellobiose and gentiooligosaccharides, and/or

at least one NDP selected from the group consisting of beta-glucans, xylans, arabinoxylans, arabinogalactans, arabinogalactanpeptides, xyloglucans, mannans, galactomannans, and cellulose, and from 0 to 2% by weight glucose and 0 to 5% by weight of starch.

In a preferred embodiment, the NDO is an oligosaccharide having a degree of polymerization ranging from 2 to 10.

In a preferred embodiment, the NDP is a polysaccharide having a degree of polymerization superior to 10.

One embodiment of the present invention concerns a process wherein the food composition obtained comprises at least one NDO in a concentration by weight ranging between 1 and 85%; and/or at least one NDP in a concentration by weight ranging between 1 and 85%, and gluconic acid and/or a salt thereof in a concentration by weight ranging between 10 and 50%.

One embodiment of the present invention concerns a composition wherein, said dietary fiber comprises at least NDO and at least one NDP.

In an embodiment, said NDO is selected from the group consisting of arabinoxylooligosaccharides, xylooligosaccharides, beta-glucan oligosaccharides, cellobiose, organic isomaltooligosaccharides and mixture thereof. In another embodiment, said NDP is selected from the group consisting of arabinoxylans, arabinogalactans, arabinogalactanpeptides, beta-glucans, and mixtures thereof. Preferably, the composition according to the invention comprises at least one NDO selected from the group consisting of arabinoxylooligosaccharides, xylooligosaccharides, beta-glucan oligosaccharides, cellobiose, organic isomaltooligosaccharides and mixture thereof, and at least one NDP selected from the group consisting of arabinoxylans, arabinogalactans, arabinogalactanpeptides, beta-glucans, and mixtures thereof. Preferably, the composition according to the invention comprises gluconic acid or a salt thereof, at least one NDO which is arabinoxylooligosaccharides, and at least one NDP which arabinoxylans. Preferably, the composition according to the invention comprises selenium gluconate, at least one NDO which is arabinoxylooligosaccharides, and at least one NDP which arabinoxylans.

According to an embodiment, said at least one NDO is cellobiose or isomaltooligosaccharide. Preferably said isomaltooligosaccharide is organic isomaltooligosaccharide.

Table 1 lists the structure and origin of non-limiting examples of suitable NDO for use in the present invention.

TABLE 1

| Name | Structure | Origin | Trade name |
|---|---|---|---|
| Xylooligosaccharides (XOS) | Xₙ with β (1-4) | Cereals, partial | Xylooligo ®^{,} |
| or | bonds, more or | hydrolysis of xylan | |
| Arabinoxylooligosaccharides | less substituted or | or arabinoxylan | |
| (AXOS) | not by A on O-2 | containing material | |
| | and/or O-3, | (corn cobs, cereal | |
| | | grain hemicellulose, | |
| | | . . .) | |
| Arabinogalactanoligosaccharides | Ga-oligomer with β | Cereals, extraction, | |
| | (1-3) bonds | partial hydrolysis of | |
| | substituted by A | arabinogalactans | |
| | and Ga on O-4 | | |
| | and O-6 | | |
| Isomaltooligosaccharides | Gₙ with α(1-6), α | Starch, partial | Isomalto ® |
| (IMO) | (1-2), or α (1-3) | enzymatic hydrolysis + | |
| | bonds | glucosyltransferase | |
| Xyloglucan oligosaccharides | G-polymer with β | Cereals, extraction, | |
| | (1-4) bonds | partial hydrolysis | |
| | substituted by X | | |
| | on 0-6 | | |
| Galactomannan | M-oligomers with β | Plants, Guar or cluster | |
| oligosaccharides | (1-4) or β (1-3) | bean, partial hydrolysis | |
| | bonds and | | |
| | substituted by | | |
| | galactose on O-5 | | |
| Mannans oligosaccharides | M-oligomers with β | Plants, Guar or cluster | |
| | (1-4) or β (1-3) | bean, partial hydrolysis | |
| | bonds | | |
| Resistant dextrin | G-oligomer, | Wheat or maize starch, | Nutriose ®, |
| glucooligosaccharides | randomly | dextrinization and | Fibersol ® |
| | branched with α | repolymerization | |
| | and β (1-4), (1-6), | | |
| | (1-3) and (1-2) | | |
| | bonds | | |
| β-glucan | G-oligomer with β | Cereals, extraction, | |
| glucooligosaccharides | (1-3) and β (1-4) | partial hydrolysis of β- | |
| | bonds | glucan | |
| Cellobiose | G₂ with β (1-4) | Cellulosic material, | |
| | | partial hydrolysis | |
| Cellulooligosaccharides | Gₙ with β (1-4) | Cereals, partial | |
| (COS) | bonds | hydrolysis of cellulose | |
| Gentiooligosaccharides | Gₙ with β (1-6) | Starch, enzymatic | |
| (GeOS) | bonds | reaction | |

G = glucose, X = xylose, M = mannose; n = number of monosaccharide units

Table 2 lists the structure and origin of non-limiting examples of suitable NDP for use in the present invention.

TABLE 2

| Name | Structure | Origin | Trade name |
|---|---|---|---|
| β-glucans | G-polymer with β (1- | Cereals, extraction | OatVantage ®' |
| | 3) and β (1-4) bonds | | Barliv ® |
| Arabinoxylans or Xylans | X-polymer with β (1- | Cereals, extraction | |
| | 4) bonds substituted | | |
| | or not by A on O-2 | | |
| | and/or O-3 | | |
| Arabinogalactans | Ga-polymer with β | Cereals, extraction | |
| | (1-3) bonds | | |
| | substituted by A and | | |
| | Ga on O-4 and 0-6 | | |
| Arabinogalactanpeptides | Peptidic polymer | Cereals, extraction | |
| | substituted with β | | |
| | arabinogalactans | | |
| Xyloglucans | G-polymer with β (1- | Cereals, extraction | |
| | 4) bonds substituted | | |
| | by X on 0-6 | | |
| Galactomannans | M-polymers with β | Plants, Guar or | |
| | (1-4) or β (1-3) | cluster bean | |
| | bonds and | | |
| | substituted by | | |
| | galactose on O-5 | | |
| Mannans | M-polymers with β | Plants, Guar or | |
| | (1-4) or β (1-3) | cluster bean | |
| | bonds | | |
| Cellulose | G-polymer with β (1- | Several | Avicell ® |
| | 4) bonds | lignocellulosic | |
| | | materials, | |

G = glucose, X = xylose, A = arabinose, Ga = galactose, M = mannose

In view of the prebiotic effects of the NDO and/or NDP, the composition obtained according to the process of the invention, is useful for providing a technical, nutritional and/or health benefit to an individual in need thereof. In an embodiment, the present composition can be used for the selective stimulation of the growth and/or activity of the gastro-intestinal microflora. In a further embodiment, said composition can also be used for the alleviation of constipation, improving gut health, improving mineral absorption, or an improving lipid metabolism and a better regulation of glycemia/insulinemia. The present composition can also be used for the reduction of the risk of heart disease, diabetes and/or metabolic syndrome, cancer prevention, positive impact on hepatic encephalopathy, immunomodulation, inflammation reduction. The present composition is also particularly useful for improving satiety.

The present invention also provides a composition suitable for a functional food additive composition, and preferably as a prebiotic composition, comprising:
gluconic acid and/or a salt thereof in a concentration by weight ranging between 1 and 60%; preferably 11 to 50%, for example 15 to 50%,
at least one NDO selected from the group consisting of xylooligosaccharides, arabinoxylooligosaccharides, beta-glucan glucooligosaccharides, arabinogalactanoligosaccharides, isomaltooligosaccharides, xyloglucan oligosaccharides, galactomannan oligosaccharides, mannan oligosaccharides, cellulooligosaccharides, cellobiose and gentiooligosaccharides in a concentration by weight ranging between 1 and 95%, preferably 5 and 85%, for example between 5 and 50%, and optionally
at least one NDP selected from the group consisting of beta-glucans, xylans, arabinoxylans, arabinogalactans, arabinogalactanpeptides, xyloglucans, mannans, galactomannans and cellulose in a concentration by weight ranging between 0 to 95%, preferably 1 and 95%, preferably from 0 to 20% and more preferably from 5 to 20%.

In an embodiment, said composition comprises:
gluconic acid and/or a salt thereof in a concentration by weight ranging between 1 and 60%; preferably from 11 to 50%,
at least one NDO selected from the group consisting of xylooligosaccharides, arabinoxylooligosaccharides, beta-glucan glucooligosaccharides, arabinogalactanoligosaccharides, isomaltooligosaccharides, xyloglucan oligosaccharides, galactomannan oligosaccharides, mannan oligosaccharides, cellulooligosaccharides, cellobiose and gentiooligosaccharides in a concentration by weight ranging between 1 and 95%, preferably from 5 to 85% and
at least one NDP selected from the group consisting of beta-glucans, xylans, arabinoxylans, arabinogalactans, arabinogalactanpeptides, xyloglucans, mannans, galactomannans and cellulose in a concentration by weight ranging between 1 and 95%, preferably from 5 to 20%.

The NDO and NDP for use in the present composition can be either extracted from natural sources, obtained by enzyme processing, and/or produced chemically. For example NDP such as plant cell wall constituents or hemicelluloses can be used but also synthetic NDP and NDO can be used which are mainly but not exclusively produced starting from starch.

In an embodiment, the NDO and NDP for use in the present composition are originated from plant based material wherein the plant is selected from the group consisting of cereals, legumes, tubers and mixtures thereof.

The composition according to the present invention comprises gluconic acid, a monomeric organic acid (DP 1) that is not a carbohydrate, and further at least one NDO (DP 2-10) and at least one NDP (DP>10), resulting in a functional food additive with a well-balanced distribution of chain lengths.

Gluconic acid and/or a salt thereof is present in the composition in a concentration by weight ranging between 1 and 60%, preferably between 10% and 50%, preferably between 11% and 50%, preferably between 15% and 50%, and most preferably between 20 and 40%.

The NDO in said composition is preferably selected from the group consisting of xylooligosaccharides, arabinoxylooligosaccharides, beta-glucan glucooligosaccharides, arabinogalactanoligosaccharides, isomaltooligosaccharides, xyloglucan oligosaccharides, galactomannan oligosaccharides, mannan oligosaccharides, cellulooligosaccharides, cellobiose and gentiooligosaccharides and is present in the composition in a concentration by weight ranging between 1 and 95%, preferably between 5 and 90%, preferably 5 and 85%, and most preferably between 10 and 80%. The NDP in said composition is preferably selected from the group consisting of beta-glucans, xylans, arabinoxylans, arabinogalactans, arabinogalactanpeptides, xyloglucans, mannans, galactomannans and cellulose in a concentration by weight ranging between 0 and 95%, preferably between 0 and 20%, preferably between 1 and 95%, preferably between 5 and 80%, preferably between 5 and 20%, between 10 and 50% and most preferably between 10 and 20%.

In a preferred embodiment, said NDO is selected from the group consisting of arabinoxylooligosaccharides, xylooligosaccharides, beta-glucan glucooligosaccharides, cellobiose, organic isomaltooligosaccharides and mixtures thereof. Preferably the NDO is selected from arabinoxylooligosaccharides.

In another preferred embodiment said NDP is selected from the group consisting of arabinoxylans, arabinogalactans, arabinogalactanpeptides, beta-glucans, and mixtures thereof. Preferably NDP is selected from arabinoxylans.

In a further preferred embodiment, the NDO is selected from the group consisting of xylooligosaccharides, arabinoxylooligosaccharides, beta-glucan glucooligosaccharides, and mixtures thereof, and the NDP is selected from the group consisting of arabinoxylans, arabinogalactans, arabinogalactanpeptides, beta-glucans and mixtures thereof. In a further preferred embodiment, the NDO is selected from arabinoxylooligosaccharides, and the NDP is selected from arabinoxylans.

In yet another embodiment, the NDO is cellobiose or organic IMO.

In another embodiment, the composition according to the present invention may further comprise 1 to 60% by weight of inulin and/or oligofructose. Preferably the composition may comprise from 5 to 50%, preferably from 10 to 40%, more preferably from 20 to 30% by weight of inuline.

The composition can be formulated as a powder, a liquid or a dispersion of a powder in a liquid.

The presence of gluconic acid together with the NDO and NDP covers surprisingly the bitter taste, and/or after taste, which is characterized by a “vegetal taste” associated with NDO and/or NDP originated from plant based material.

The presence of gluconic acid with some NDO and NDP also allows in some specific cases, the present composition to be available under a liquid form such as a syrup formulation. In prior art formulation, the presence of the longest chains with low solubility does not allow a concentration in the final solution that is compatible with a stable syrup formulation. For instance, mixture of arabinoxylooligosaccharides (NDO) and arabinoxylans (NDP) is polydisperse. The dry matter concentration that is required for a good natural osmotic preservation is such that the syrup becomes too viscous, and is not suitable for syrup formulation. The present composition with the presence of gluconic acid and/or a salt thereof, allows to overcome this problem and to obtain a syrup with a reduced dry matter concentration.

Gluconic acid in the present invention is useful as water activity reducer for the composition. The higher moisture content of the present composition reduces the viscosity of the final solution, which is compatible with syrup formulation that contains molecules with high degree of polymerization. Therefore, gluconic acid with its preservative effect allows the production of a syrup formulation with a dry matter content that would not have been sufficient for a good preservation, if the solution was only composed of NDO and NDP.

Another surprising advantage of the produced composition according to the present invention is a positive effect on the glass transition temperature (Tg). For example, the combination of gluconic acid with arabinoxylan and arabinoxylooligosaccharides or with inulin reduces the Tg of the composition by a few degrees (° C.) (cfr Table 3 in example 10). Without being bound to any theory this is believed to be due to a plasticizing activity of gluconic acid. It was surprisingly found that gluconic acid can acts as a plasticizer. This is of particular interest in food preparations where a plasticizing effect is highly desired such as (not limiting) cereal bars, cookies, biscuits, confectionery products, ice creams, and the like.

The compositions according to the invention are particularly useful as food additive, in particular as functional food additive and preferably as prebiotic composition.

The present invention therefore also encompasses the use of a composition according to the present invention as functional food additive.

The use of the compositions according to the present invention indeed provides several nutritional and/or health benefits, due to the presence of gluconic acid associated with the presence of NDP and NDO.

The selective fermentation in the gut by one or more health-promoting micro-organisms like Bifidobacteria or Lactobacilli is called the prebiotic effect. The specificity of gluconic acid, being a prebiotic non-carbohydrate monomeric organic acid is believed confer synergistic effects with NDO and NDP with regard to a number of health benefits cited below.

The composition according to the present invention, by its combination of gluconic acid with at least one NDO and NDP can be used for its prebiotic effect. In fact, the synergistic effects are most likely linked to the balanced distribution of chain lengths in the functional food additive: a non-carbohydrate monomeric organic acid (DP 1) in combination with one or more non-digestible oligosaccharides (DP 2-10) and one or more non-digestible polysaccharides (DP>10). These different types of molecules with different chain lengths can be fermented by different types of beneficial micro-organisms and/or at different locations in the large intestine. In this way, different benefits of different types of beneficial micro-organisms could be combined (for instance production of butyrate, vitamin production, production of antimicrobial substances, . . . ) and/or benefits could be distributed all along the large intestine, considering the fact that the smaller molecules (DP=<10), and specially gluconic acid (DP=1), are degraded preferentially in the proximal part and the larger molecules (DP>10) in the more distal part of the colon.

The composition according to the present invention can be useful for providing a technical, nutritional and/or health benefit to an individual in need thereof. Said composition can be used for the selective stimulation of the growth and/or activity of the gastro-intestinal microflora. In a further embodiment, said composition can also be used for the alleviation of constipation, for improving gut health, for improving mineral absorption, for improving lipid metabolism and/or for a better regulation of glycemia/insulinemia. The present composition can also be used for the reduction of the risk of heart disease, diabetes and/or metabolic syndrome, cancer prevention, positive impact on hepatic encephalopathy, immunomodulation, inflammation reduction. The present composition is also particularly useful for improving satiety.

For example, the presence of inulin together with gluconic acid in the composition can be favorable for a higher proportion of butyric acid in the short chain fatty acid pool produced by the colon fermentation, which is favorable for the health of the colonocytes.

The presence in the composition according to the invention of a mixture of a non-carbohydrate monomeric organic acid (DP 1) and further NDO and NDP of different types and/or different chain length may be considered as optimal concerning health benefits. The action of the different chain length molecules can thus be progressive along the colon, the shortest chains acting first, in the most proximal part of the colon, the longest chains, acting in a more distal part of the colon. This results in the stimulation of beneficial bacteria and the production of short-chain fatty acids all along the complete trajectory of the colon and a corresponding overall reduction of the pH of the colon. Due to the lower pH, uptake of calcium and other minerals is improved all along the colon.

The presence of gluconic acid in the form of calcium gluconate in the composition is also the best way to bring calcium into the colon where it can play its physiological role and be absorbed for a better calcium balance of the host.

Other potential health benefits of compositions comprising gluconic acid and further at least one NDP and at least one NDO closely relate to the prebiotic effect and include alleviation of constipation, increasing fecal bulk, improving large bowel function, improved mineral absorption, reducing plasma cholesterol concentrations, improved lipid metabolism and thus reduction of the risk of heart disease and/or metabolic syndrome, cancer prevention, impact on hepatic encephalopathy, glycemia/insulinemia regulation, and immunomodulation. Another interesting potential health benefit that is of first interest in the fight against obesity, is to act on the feeling of fullness or satiety through the regulation of gut peptides like GLP-1, PYY or ghrelin.

The present invention also encompasses a method for preparing a food product or beverage comprising the steps of:

a. providing a composition obtained according to the process of the present invention, or a composition according to the present invention, and

b. formulating said composition into a food product, a feed product or a beverage.

The present invention also concerns a food product containing the composition according to the present invention, as well as a feed product containing the same composition and a beverage containing the same.

The following examples illustrate the present invention.

### EXAMPLES

In examples 1 to 8, 12, 13 different embodiments of process and compositions according to the invention are presented and illustrated by different steps and products. Example 9 illustrates the plasticizing effect of gluconic acid. Examples 10 and 11 illustrate the prebiotic effect of two compositions according to embodiments of the present invention.

### Example 1

### Production of a Slurry Containing Glucose and Arabinoxylan (Steps (a) and (b) of the Process According to an Embodiment of the Invention)

A three phase decanter overflow coming from a starch gluten separation process, was analyzed with the following results: pH: 5.5; Dry matter (D.M.)=9.8%; starchy material+glucose=50% on D.M.; Arabinoxylans=21% on D.M.; proteins (Dumas)=20% on D.M; others 9% on D.M.

The product is heated to about 100° C., cooled down to about 90° C., alpha-amylase is added, and the slurry is sent to a storage tank for about 3 hours. After centrifugation, the overflow of the sludge separator is then again heated to about 100° C. After cooling down to about 80° C., beta-amylase is added and after about 3 hours, the slurry is cooled down and maintained at about 55° C. At that temperature, glucoamylase, alcalase, and alkaline protease are added and the product is sent to a storage tank for about 12 hours. Thereafter, the product is filtered with expanded Perlite as filter aid, heated again to about 85° C. and cooled down in a cooling and storage room. The product is analyzed with the following results: pH 5.5; D.M.=10%; arabinoxylans=18.5% on dry matter; free glucose=47% on D.M.; proteins (Dumas)=11% on D.M.

### Example 2

### Production of a Syrup Containing Gluconic Acid, Arabinoxylan, and Arabinoxylooligosaccharides (Step (c) and (d) of the Process According to an Embodiment of the Invention)

One liter of a product prepared in example 1 is heated to about 20° C., passed up-flow across a strong cation regenerated (H form) exchange resin, than, down-flow across a weak anion regenerated (OH form) resin. The protein content after this ionic exchange is 1.5% on D.M.

This demineralized product is heated to about 50° C., pH is adjusted to about 7.5 by addition of sodium hydroxide, and the product is added to a reaction vessel with a rotating agitator at 600 rpm. Air is added at a rate of 7 liters of air per minute. 100 U enzyme of Glucose oxidase (Gluzyme™ 10 000BG from Novo Nordisk) per gram of glucose is added, followed by the addition of 1 000 Units of catalase (Catazyme™ 25L from Novo Nordisk) per gram of glucose. During the reaction time, pH evolution is monitored and adjusted when necessary by sodium hydroxide addition to restore the initial set point pH of 7.5+/−0.2. After about 6 hours reaction time, 50% of the present glucose is oxidized to gluconic acid ; after about 12 hours, the glucose is totally converted to gluconic acid.

The product is then passed, at room temperature, down-flow across a couple of regenerated (H) strong cation, (OH) weak anion exchange resins, and leakage of gluconic acid is allowed until the total effluent contains about 30% of gluconic acid on D.M.

The pH is then adjusted to pH about 4.5, by addition of calcium hydroxide and the temperature is increased to about 55° C., enzyme (Shearzyme™ 2× from Novo Nordisk, 0.03% enzyme on arabinoxylan dry matter) is added under continuous stirring to partially convert arabinoxylan to arabinoxylooligosaccharides. The hydrolysis of arabinoxylan is stopped after about 12 hours.

By concentration under vacuum the product is brought to about 50% D.M., and is as such suitable for commercialization under fridge stable syrup form. This syrup can also be spray dried to obtain a stable powder. The obtained syrup contains on D.M: about 30% gluconic acid, a total content of about 60% dietary fiber from which about 65% as arabinoxylooligosaccharides (DP 2-10) and about 35% as arabinoxylan (DP comprised between 11 and about 250).

### Example 3

### Purification of Arabinoxylan and Arabinoxylooligosaccharides: Elimination of Gluconate by Calcium Precipitation (Step (d) of the Process According to an Embodiment of the Invention)

One liter of the product prepared following example 1 is adjusted to pH of about 5 by addition of hydrochloric acid, the temperature is increased to 55° C., and enzyme (Shearzyme 2× from Novo Nordisk, 0.03% enzyme on AX dry matter) is added under continuous stirring to partially convert arabinoxylan to arabinoxylooligosaccharides. The hydrolysis of arabinoxylan is stopped after about 12 hours. Then the product is heated to about 50° C., pH is adjusted to about 7.5 by addition of calcium hydroxide, and the product is put in a reaction vessel with a rotating agitator at about 600 rpm. Air is added at a rate of 7 liters of air per minute. 100 U enzyme Glucose oxidase (Gluzyme^{TM} 10 000BG from Novo Nordisk) per gram of glucose is added, followed by the addition of 1 000 Units of catalase (Catazyme™ 25 L Novo Nordisk) per gram of glucose. During the reaction time, pH evolution is monitored and corrected when necessary by calcium hydroxide addition to restore the initial set point of pH 7.5+/−0.2. After about 6 hours reaction time about 50% of the present glucose is oxidized to gluconic acid; after about 12 hours, the present glucose is totally converted to gluconic acid.

The reaction medium is then vacuum concentrated to about 30% D.M. at about 60° C. The mixture is slowly cooled down to about 20° C. (takes about 2 hours) under gently stirring. The calcium gluconate precipitation is achieved after about 12 hours. The mixture is then centrifuged and the solid calcium gluconate discarded. The supernatant contains about 15% calcium gluconate and about 30% arabinoxylooligosaccharides and arabinoxylan by weight on dry matter basis and no more glucose nor starchy material.

### Example 4

### Production of Organic IMO Syrups Starting from Organic Rice Flour

Examples 4, 5, and 6 describe the application of the process according to an embodiment of the present invention on a reaction medium based on organic rice flour. These examples were also performed on industrial scale using organic wheat starch and organic manioc starch (Data not shown).

In a 25 000 l vessel, 8400 kg rice meal was mixed with 15 200 liters water. pH was adjusted to 5.6-6.1. Alpha amylase (Termamyl from Novo) was added and liquefaction took place by gradually increasing the temperature to 90-100° C. in order to obtain a slurry with a dextrose equivalent between 8-35.

The slurry was then cooled down to 55-65° C., pH adjusted to 5.3-5.7, and beta amylase (Diazyme BB from Danisco) and Transglucosidase (L-500 from Danisco) were then added. The IMO molecules formation was followed by analysis and when more than 40% of the carbohydrates were converted to IMO the reaction was stopped by heating the slurry to a temperature higher than 70° C. Further purification and filtration steps can then be applied as usually applied in glucose syrup plants, and the purified stream was concentrated by a multistage evaporator at 80% D.M. Syrups produced following this method contained about 15 to 25 g of glucose and 10 to 20 g of digestible maltooligosaccharides with low DP values per 100 g D.M.

### Example 5

### Oxidation of Glucose in an IMO Syrup Obtained from Organic Rice Flour (Step (c) of the Process According to an Embodiment of the Invention)

In some case, the use of chromatographic methods or ion exchange resins does not fit with the “organic” specifications: the present example offers a technological solution for this limitation.

A product obtained as in example 4 was then adjusted to 30% D.M. and the reaction medium was heated to 35-55° C., with a pH adjusted at approximately 7.5. Glucose oxidase (Gluzyme from NOVO) and catalase (Catazyme 25 L from Novo) were added in appropriate concentrations between 30-100 and 300-1000 U/g of glucose, respectively. Air was injected at a flow rate of 100 to 150 liters per minutes per kg of glucose to be oxidized. Optimal pH was controlled by addition of calcium carbonate, calcium hydroxide, magnesium hydroxycarbonate or sodium hydroxide. After 18 hours the solution contained less than 1% of glucose on D.M. and about 15 to 25% gluconic acid. This product was then ready for concentration in a multistage vacuum evaporator to give a syrup with only 10 to 20% digestible carbohydrates on D.M.

### Example 6

### Production of Organic IMO Syrups from Organic Rice Flour with Specific Hydrolysis of Digestible Oligosaccharides and Elimination of Produced Glucose. (Steps (b), (c), and (d) of the Process According to an Embodiment of the Invention)

A product obtained as in example 4 is adjusted to 30% D.M. and a specific hydrolysis of digestible oligosaccharides has then been carried out. Starting from the non reducing end of the untransglucosylated saccharides, α-(1-4) bounds was subjected to hydrolysis by a specific amylase from the alpha-glucosidase (EC 3.2.1.20) or amyloglucosidase (EC 3.2.1.3) family. This step allowed to obtain an IMO solution where glucose became nearly the only impurity. The glucose content of this medium was about 25 to 45% on D.M.

This product can then be processed as in example 4. Moreover, because of the almost absence of digestible oligosaccharides, the product contained between 25 and 45% gluconic acid after the glucose oxidation step. Optionally a part of the gluconic acid can be eliminated by filtration after precipitation with calcium carbonate, calcium hydroxide or magnesium hydroxycarbonate. On a practical point of view, this precipitation step can be coupled with the pH control during the glucose oxidation using these three alkaline agents. Using this last step, the product obtained has an IMO content on D.M. of nearly 100% and can be further concentrated and/or spray dried.

### Example 7

### Production of Cellobiose and Gluconic Acid from Cellulosic Material

7.1: Saccharification Process.

In a 250 ml flask, cellulose (12.5 g) is suspended in a citrate buffer (250 mL, 0.05N, pH 4.85) under magnetic agitation then heated to 50° C. Cellulase from Trichoderma reesei QM9414 (298 μL, 57 UFP/mL, 1.3 UPF/g cellulose) is added. After 6 hours the suspension is cooled to room temperature and then filtered. The sweated filtrate solution (230 mL) is collected for the oxidation step. The wet solid residue, after weight/weight analysis, is suspended (5% w/vol) in a citrate buffer and maintained to 50° C. to perform a second cellulolytic hydrolysis. After 6 hours the suspension is cooled to room temperature, filtered and treated as above. The same procedure as above is repeated twice.

This original and continuous process allows to produce without pretreatment 494mg of cellobiose and 109 mg of glucose per 1 g of starting cellulose.

7.2: Oxidation Process.

A citrate buffer solution (500 mL) containing glucose (1 g/L) and cellobiose 8 g/L), is adjusted to pH 6.4 by adding an aqueous solution of sodium hydroxide 1N (25 mL). The whole is heated to 35° C. under vigorous stirring then glucose oxidase/catalase solution (40 μL, 225 U/2250 U/g glucose, Hyderase L from Amano) is added and an air flux (3 L/minute) was maintained for 7 hours to have a complete oxidation of glucose without oxidation of cellobiose.

### Example 8

### Composition of a Mixture for a Better Management of the Bone and Gut Health

100 g of the powder obtained following example 2 is mixed with 40 g pure inuline.

This mixture contains on D.S.: 21% gluconic acid, 29% inuline, about 28% arabinoxylooligosaccharides (DP=<10) and about 15% arabinoxylan, (10<DP<250). This mixture is a typical composition of a food additive to be added in yoghurt at the rate between 1 and 5 g food additive for 100 g yoghurt.

### Example 9

### Plasticizing Effect of Gluconic Acid

A mixture containing 12 g calcium gluconate and 28 g of a mixture of arabinoxylooligosaccharides (AXOS) and arabinoxylan was produced by the process described in this invention and was lyophilized and stabilized at 100% dry matter by equilibrating it with a P₂O₅ salt in a closed vessel. A sample of the obtained powder was submitted to the analysis of the glass transition temperature (Tg) by means of the differential scanning calorimetry (DSC) equipment. The reduction in Tg of the arabinoxylooligosaccharides/arabinoxylan mixture is presented in Table 3.

A mixture containing 12 g calcium gluconate and 28 g inuline was lyophilized and stabilized as above. The Tg of the obtained powder was measured as above. The reduction in Tg is given in table 3.

TABLE 3

| | | inulin | AXOS/arabinoxylan mixture** |
|---|---|---|---|
| Pure product | Tg | 92 | 150 |
| | Std dev* | 3 | 2.3 |
| With 30% Gluconic | Tg | 85 | 142 |
| | Std dev* | 4 | 1 |

*standard deviation on 3 measurements
**with DP from 3 to about 250

### Example 10

### In Vitro Prebiotic Effect of a Composition Containing 40% by Weight Calcium Gluconate and 60% by Weight of a Mixture of Arabinoxylooligosaccharides and Arabinoxylans with DP of the Mixture from 3 to about 250

The prebiotic effect of a composition containing 40% by weight calcium gluconate and 60% by weight of a mixture of arabinoxylooligosaccharides and arabinoxylans with a DP of the mixture from 3 to about 250 is measured as follows.

An in vitro model described by Bindelle et al (2007, Animal feed Science and Technology 132, 111-122) is used. The fermentation inoculum is constituted of the colonic content sampled from 3 growing pigs that are canulated at 20 cm of the ceco-colonic junction. The animals are individually housed and fed ad libitum with a commercial feed adapted to their age. The colonic contents of the 3 pigs are mixed together. The digestive content is mixed with a buffer solution at a ratio of 0.1 g/ml. Each test is conducted on 200 mg of test fiber sample is added. Gas formation is followed in function of time. Fermentation kinetics are determined. Short-chain fatty acids are determined according to Bindelle et al (2007, Animal 18, 1126-1133).

The short-chain fatty acids content is significantly increased as compared to a standard cellulose fiber.

### Example 11

### In Vivo Prebiotic Effect of a Composition Containing 25% by Weight Calcium Gluconate and 25% by Weight of a Mixture of Arabinoxylooligosaccharides and Arabinoxylans with a DP of the Mixture from 3 to about 250, and 50% by Weight of Inulin

The in vivo prebiotic effect of a composition containing 25% by weight calcium gluconate and 25% by weight of a mixture of arabinoxylooligosaccharides and arabinoxylans with a DP of the mixture from 3 to about 250, and furthermore 50% by weight of inulin, as compared to a placebo (cellulose) is measured as follows.

Growing rats are fed a standard diet corresponding to their growth needs. The above composition or the placebo is added at 7.5% by weight to the diet in replacement of starch and saccharose in order to constitute the experimental diet. The standard and experimental diets are presented in Table 4. The values in Table 4 are expressed in g DM/kg DM.

TABLE 4

| Feed Components | Standard diet | Experimental diet |
|---|---|---|
| Casein | 144.5 | 144.5 |
| DL-methionine | 5.2 | 5.2 |
| Microcristalline cellulose | 50 | 50 |
| Maize starch | 466.9 | 416.9 |
| Saccharose | 233.4 | 208.4 |
| Vitamins (vitamin diet fortification | 10 | 10 |
| mixture, MP Biomedicals) | | |
| Minerals (AIN-76, MP Biomedicals) | 40 | 40 |
| Rapeseed and peanut oil (1/1 V/V) | 50 | 50 |
| Prebiotic composition* or placebo | 0 | 75 |
| (cellulose) | | |

*composition containing 25% by weight calcium gluconate and 25% by weight of a mixture of arabinoxylooligosaccharides and arabinoxylans with a DP of the mixture from 3 to about 250, and furthermore 50% by weight of inulin

Male Wistar Han rats with initial weight of +/−50g are used. Two groups of 8 rats are individually housed in metabolic cages. The temperature is maintained at +/−22° C. and the relative humidity at +/−70%. A light cycle of 12 h is applied. After five days adaptation period to the standard diet (ad libitum) and the cages the rats are weighed. From day 5 to day 30 one group of rats is fed the standard diet, the second group is fed the experimental diet containing the prebiotic composition, at a level equal to 95% of the average ingestion level measured during the 5 days-adaptation period. Drinking water is available ad libitum.

Growth parameters: Feed intake is measured for each rat on a weekly basis by difference between the available and refused amount. The growth is determined by difference between the weight at day=n and the weight at day=n+7. The weights are recorded at fixed times without fasting period.

Microbiological parameters: On days 5, 16 and 27, Bifidobacteria and Lactobacilli counts are realized on fecal samples collected according to Ten Bruggencate et al (2005, J. Nutr. 135, 837-842). Fecal matter is quantitatively collected between day=n−1 at 9:00 AM and day=n+1 at 9:00 AM. At the end of the collection, fecal matter is lyophilized and quantified. Then they are finely ground for RT-PCR analysis according to Delroisse et al (2007, Microbiological Research doi:10.106/j.micres.2006.09.2004).

Fermentation parameters: At the end of the experience, animals are killed and the cecum is removed. The weight of the cecum and of its content is determined. The pH of the cecal content is measured, the dry matter is determined at 105° C. and the short-chain fatty acids and lactic acid are determined by liquid phase chromatography.

The experimental composition significantly modifies the fermentation pattern of the rats with an enlargement of the cecum and a decrease in cecal pH. Furthermore the amount of fatty acids is increased and the number of bifidobacteria increased.

### Example 12

### Production of Cellobiose and Gluconic Acid from Cellulosic Material

12.1: Saccharification Process.

In a 250 ml flask, 12.5 g of microcrystalline cellulose (FD-100) was suspended in citrate buffer (250 mL, 0.05M, pH 4.8) under magnetic agitation and then heated to 50° C. Cellulase from Trichoderma reesei QM9414 (17 UFP/mL, 0.4 UPF/g cellulose) was added. During the multistage experiment (four times, 6 h), hydrolysis was performed continuously for 6 h, and then the hydrolyzate was filtered with a vacuum filter funnel assembled with a fritted disk. Vacuum was run until filtrate was no longer being collected (about 10 min). Following filtration, retentates were resuspended in fresh buffer at the same concentration as in the first stage (5% w/v), at 50° C. under stirring, to continue the hydrolysis for further 6 h. Steps were repeated four times in order to complete the 24-h period. The multistage process allowed us to produce in total 2795 mg of cellobiose and 585 mg of glucose.

12.2: Oxidation Process.

A citrate buffer solution (500 mL) containing glucose (1 g/L) and cellobiose 8 g/L), was adjusted to pH 6.4 by adding an aqueous solution of sodium hydroxide 1N (25 mL). The medium was heated to 35° C. under vigorous stirring and glucose oxidase/catalase solution (40 μL, 225 U/2250 U/g glucose, Hyderase L from Amano) was then added and an air flux (3 L/minute) is maintained for 7 hours to have a complete oxidation of glucose without oxidation of cellobiose.

The product was then passed, at room temperature, down-flow across a couple of regenerated (H) strong cation, (OH) weak anion exchange resins, and the leakage of gluconic acid was tolerated until the total effluent contained 11% of gluconic acid on dry substance.

### Example 13

### Production of a Syrup Containing Gluconic Acid, Arabinoxylan, and Arabinoxylooligosaccharides Enriched with Selenium

Selenium hydroxide is added to one liter of the product prepared following example 2, to convert at least part of the gluconic acid to selenium gluconate. Selenium is useful in the present composition to prevent certain cancers such as prostate and colon cancer. Other advantage is that organic salts of selenium are more bio-available than other forms. The amount of selenium gluconate is adjusted according to the end product packaging in order to respect the selemium AJR.

1. A process for the production of a composition comprising the steps of:
a) providing a plant based material wherein the plant is selected from the group consisting of cereals, legumes, tubers and mixtures thereof, wherein said plant based material comprises dietary fiber, optionally starchy material and optionally glucose, or wherein said plant based material comprises starchy material, and optionally glucose,
b) b 1) hydrolyzing or transglucosylating at least part of the dietary fiber into glucose and into at least one non-digestible oligosaccharide, optionally into at least one non-digestible polysaccharide, and optionally hydrolyzing and transglucosylating at least part of the optional starchy material to glucose, and into at least one non-digestible oligosaccharide, or
b2) hydrolyzing and transglucosylating at least part of the starchy material to glucose and into at least one non-digestible oligosaccharide, and optionally hydrolyzing at least part of the maltooligosaccharides produced in step (b2) into glucose,
c) oxidizing at least part of the total glucose, consisting of said optional glucose of step (a) and said glucose obtained in step (b1) or (b2), to gluconic acid or a salt thereof, and
d) removing at least part of said gluconic acid or a salt thereof obtained in step (c);
thereby obtaining a composition comprising
gluconic acid or a salt thereof in a concentration by weight ranging between 11 and 50%;
dietary fiber wherein said dietary fiber comprises: at least one non-digestible oligosaccharide selected from the group consisting of xylooligosaccharides, arabinoxylooligosaccharides, beta-glucan glucooligosaccharides, arabinogalactanoligosaccharides, isomaltooligosaccharides, xyloglucan oligosaccharides, galactomannan oligosaccharides, mannan oligosaccharides, cellulooligosaccharides, cellobiose and gentiooligosaccharides in a concentration by weight ranging between 5 and 85%, and optionally at least one non-digestible polysaccharide selected from the group consisting of beta-glucans, xylans, arabinoxylans, arabinogalactans, arabinogalactanpeptides, xyloglucans, mannans, galactomannans and cellulose in a concentration by weight ranging between 0 and 20%,
optionally glucose in a concentration by weight ranging between from 0 to 2%; and
optionally starchy material in a concentration by weight ranging between from 0 to 5%.

2. The process according to claim 1, further comprising the step of hydrolyzing at least part of non-digestible polysaccharide comprised in the dietary fiber into non-digestible oligosaccharide, wherein said hydrolyzing step is performed before, between, or after any of said steps (a) to (d).

3. The process according to claim 1, wherein said steps (a), (b), (c) and (d) are performed consecutively.

4. (canceled)

5. The process according to claim 1, wherein the composition obtained comprises gluconic acid or a salt thereof in a concentration by weight ranging between 11 and 50%;
arabinoxylooligosaccharides in a concentration by weight ranging between 5 and 85%,
and optionally arabinoxylans in a concentration by weight ranging between 0 and 20%;
optionally glucose in a concentration by weight ranging between from 0 to 2% ; and
optionally starchy material in a concentration by weight ranging between from 0 to 5%.

6. A composition suitable for a functional food additive composition comprising:
gluconic acid or a salt thereof in a concentration by weight ranging between 11 and 50%;
at least one non-digestible oligosaccharide selected from the group consisting of xylooligosaccharides, arabinoxylooligosaccharides, beta-glucan glucooligosaccharides, arabinogalactanoligosaccharides, isomaltooligosaccharides, xyloglucan oligosaccharides, galactomannan oligosaccharides, mannan oligosaccharides, cellulooligosaccharides, cellobiose and gentiooligosaccharides in a concentration by weight ranging between 5 and 85%, and optionally
at least one non-digestible polysaccharide selected from the group consisting of beta-glucans, xylans, arabinoxylans, arabinogalactans, arabinogalactanpeptides, xyloglucans, mannans, galactomannans and cellulose in a concentration by weight ranging between 0 and 20%;
optionally glucose in a concentration by weight ranging between 0 and 2%, and
optionally starchy material in a concentration by weight ranging between 0 and 5%.

7. The composition according to claim 6, wherein said non-digestible oligosaccharide and non-digestible polysaccharide are originated from plant based material wherein the plant is selected from the group consisting of cereals, legumes, tubers and mixtures thereof.

8. The composition according to claim 6, wherein said non-digestible oligosaccharide is selected from the group consisting of arabinoxylooligosaccharides, xylooligosaccharides, beta-glucan glucooligosaccharides, cellobiose, organic isomaltooligosaccharides and mixtures thereof.

9. The composition according to claim 6, wherein said non-digestible polysaccharide is selected from the group consisting of arabinoxylans, arabinogalactans, arabinogalactanpeptides, beta-glucans, and mixtures thereof.

10. The composition according to claim 6, wherein the concentration by weight of gluconic acid or a salt thereof is ranging between 15 and 50%, the concentration by weight of non-digestible oligosaccharide is ranging between 10 and 50%, and the concentration by weight of non-digestible polysaccharide is ranging between 0 and 20%.

11. The composition according to claim 6, comprising gluconic acid or a salt thereof; arabinoxylooligosaccharides and optionally arabinoxylans.

12. The composition according to claim 6, wherein said composition further comprises inulin and/or oligofructose.

13. The composition according to claim 6, wherein said gluconic acid salt is selected from sodium gluconate, potassium gluconate, calcium gluconate, magnesium gluconate, iron gluconate, selenium gluconate, copper gluconate or zinc gluconate.

14. A food or prebiotic composition comprising the composition according to claim 6 as additive.

15. A method for providing a technical, nutritional and/or health benefit to a human being or animal in need thereof comprising administering the composition of claim 14 in an amount sufficient to provide a technical, nutritional and/or health benefit to the human being or animal.

16. The method according to claim 15, wherein the technical, nutritional and/or health benefit is selected from the group consisting of the selective stimulation of the growth and/or activity of the gastro-intestinal microflora, the alleviation of constipation, the improvement of gut health, the improvement of mineral absorption, improvement of lipid metabolism, the regulation of glycemia/insulinemia, the reduction of the risk of heart disease, diabetes and/or metabolic syndrome, the reduction of the risk of cancer, for use in positive impact on hepatic encephalopathy, immunomodulation, and inflammation reduction.

17. A method for providing cations to a human being or an animal in need thereof comprising administering the composition of claim 13 in an amount sufficient to provide cations to the human being or animal.

18. A method for improving satiety in a human being or animal in need thereof comprising administering the composition of claim 14 in an amount sufficient to improve satiety in the human being or animal.
